(19) United States
(12) Patent Application Publication
(10) Pub. No.: US 2021/0252224 A1
(43) Pub. Date: Aug. 19, 2021

(54) METHOD AND APPARATUS

(71) Applicant: Pacifi Limited, Cobham (GB)

(72) Inventor: Gareth Pearce, Cobham (GB)

(73) Assignee: Pacifi Limited, Cobham (GB)

(21) Appl. No.: 17/259,227

(22) PCT Filed: Jul. 10, 2019
(86) PCT No.: PCT/GB2019/051935

§ 371 Date: Jan. 11, 2021

(30) Foreign Application Priority Data
Jul. 10, 2018 (GB) 1811310.0
Jul. 5, 2019 (GB) 1909714.6

(51) Int. Cl. A61M 5/20; A61M 5/31; A61J 1/06; A61M 5/19

(52) U.S. Cl. CPC A61M 5/2046; A61M 5/19; A61J 1/065; A61M 5/3129

(57) ABSTRACT

A single use syringe barrel comprising a single integrated glass body forming a medicament chamber, a propellant chamber, and a glass wall separating the medicament chamber from the propellant chamber.

### FIELD OF THE INVENTION

The present disclosure relates to packaging, and more particularly to methods and apparatus for packaging compositions such as medicaments, and still more particularly to methods and apparatus for packaging comprising a piston for expelling a packaged substance from the packaging.

### BACKGROUND

Pre-filled syringes are increasingly popular. They offer a particularly convenient vehicle for delivery of unit dose medication. They minimize drug waste, and can increase product life span, while patients are able to self-administer injectable drugs at their home instead of the hospital.

Plastic syringes are widely used in a range of therapeutic sectors, such as vaccines, blood stimulants, and therapeutic proteins, however their barrier properties to gasses and moisture are often inadequate.

An auto-injector is a medical device designed to deliver a dose of a particular drug by automatic injection. Most auto-injectors comprise spring-loaded syringes intended for self-administration by patients, or administration by untrained personnel. It has also been proposed to use a propellant, such as compressed gas, to expel a dose of a drug from such a syringe for injection into a patient.

Such prior art systems give rise to a variety of problems.

### SUMMARY

Aspects and examples of the invention are set out in the claims, and aim to provide improved auto-injector apparatus and components thereof.

In an aspect there is provided a syringe barrel comprising: a single integrated glass body forming a medicament chamber, a propellant chamber, and a glass wall separating the medicament chamber from the propellant chamber.

A syringe apparatus is also disclosed. This may provide a dual chamber syringe, which may comprise two pistons, and at least one bypass for allowing fluid to flow from one chamber of the syringe, around a piston, to the other. One such syringe comprises:
a medicament chamber having an outlet to enable a medicament to be expelled from the medicament chamber;
a first piston movable to expel medicament from the medicament chamber via the outlet;
and a propellant chamber having a glass wall breakable to release propellant to move the first piston to expel said medicament.

The medicament chamber, the glass wall, and the propellant chamber may be integrally formed. For example they may be provided by a single, integral, piece of glass.

The glass wall may be weakened, for example along a cleave line which may circumscribe a region of the glass wall. The cleave line comprise etching or laser filamentation of the glass wall. The cleave line may be discontinuous.

The glass wall may comprise a shoulder, such as an annular region which may be flat and disposed at a selected angle, such as an oblique angle, to an opening of the glass body (such as a neck of the propellant chamber). This annular region may be part of a complete cone—e.g. the wall may be cone shaped, or the wall may be bulb shaped (e.g. in the manner of a vial such as a test-tube shape) and may comprise the annular region—e.g. the annular region may join the wall to the propellant chamber. The weakening referred to above may be disposed on this shoulder.

The syringe may be a tube shape, and the shoulder surface may be disposed at an oblique angle to an axis of the tube, or at right angles to it. A movable member, such as an actuator rod, may be disposed in the propellant chamber and may be actuable to break the glass wall.

The syringe may comprise a bypass channel in a sidewall wall of the medicament chamber. For example, the syringe may comprise a second piston disposed in the medicament chamber, and a bypass channel in a sidewall wall of the medicament chamber between the second piston and the outlet. A first component of a medicament composition may be disposed between the first piston and the second piston, and a second component of the medicament composition may be disposed between the second piston and the outlet. A propellant, such as HFA may be provided in the propellant chamber.

Also described herein is a method of packaging a medicament, the method comprising:
providing a syringe barrel having a propellant chamber having a glass wall, wherein a first piston is disposed in the syringe barrel;
and providing a first medicament into the syringe barrel via an open end of the barrel, between the first piston and the open end.

After providing the medicament the open end of the syringe may be closed with a stopper having an outlet for delivery of the medicament from the syringe.

The outlet may comprises a connection for connecting the stopper to a hypodermic needle or another delivery lumen such as a nozzle. Such a lumen may be carried on a hub comprising a deformable portion adapted to enable the stopper to be pierced by said lumen.

The syringe barrel may comprise a bypass channel, in which case the method can further comprise providing a second piston into the barrel, past the bypass channel to enclose the first medicament, and providing a second medicament into the barrel after the second piston.

The above described “shoulder” construction may be used in containers other than syringes. For example, an aspect of the disclosure provides a dual vial comprising a first vial and a second vial, wherein the first vial is nested inside the second vial. The second vial may have a neck joined at one end to the body of the vial and being open at its other end, e.g. to provide a mouth. The first vial also has a mouth, e.g. an opening for use in filling the first vial, and it may be otherwise closed to provide a container for holding a substance inside the first vial. The mouth of the first vial may be joined to an internal wall of the neck of the second vial, for example the join between the mouth of the first vial and the neck of the second vial may be provided by a shoulder.

This shoulder may be annular and may have flat, but sloping, surface. It may be disposed at any selected, such as an oblique angle, to the open end of the neck. In one particular example, the shoulder surface comprises a portion which is disposed at 45° to the open end of the neck and/or to an axis of the neck (e.g. where the neck is cylindrical). This annular region may be part of a complete cone—e.g. the first vial may be conical, or the annular region may provide the sloping wall of frusta-conical form, joined at its narrow end to the mouth of the first vial. In these embodiments, the first vial may be ampoule or test-tube shaped. The weakening referred to above may be disposed on this shoulder.

Thus, an embodiment of the present disclosure provides a dual vial comprising a first vial and a second vial, wherein the first vial is nested inside the second vial; wherein the first vial is joined to the second vial via a shoulder. The shoulder may be weakened, e.g. it may comprise defects such as filamentation, arranged along the length of the shoulder. The shoulder may surround a mouth of the first vial, and may join the mouth of the first vial to an internal surface of the second vial, such as around the inside surface of the neck of the second vial. The defects in the shoulder may be disposed in a continuous (e.g. closed loop) path around the shoulder. This may provide a cleave line to enable the first vial to be separated from the second vial.

This cleave line may be provided by forming weakened portions in a break zone between the first vial and the second vial. The wall of the first vial may comprises a portion having a circular cross-section, and the break zone may lie circumferentially around the wall. The weakened portions may be formed by applying a laser to the shoulder. This may cause laser filamentation within the shoulder and/or a laser ablation on and/or within the shoulder. The shoulder may be provided by a portion of the wall of the first vial. The laser may be applied to the wall of the first vial via a mouth of the second vial. The laser may be an ultra-short pulse laser.

### FIGURES

Embodiments of the present disclosure will now be described, by way of example only, with reference to the accompanying drawings, in which:

FIG. 1A shows a diagram of a single use syringe barrel having a propellant chamber, and a medicament chamber separated by a glass wall;

FIG. 1B shows a side view of a syringe barrel such as that illustrated in FIG. 1A, and a series of thumbnail sketches of possible arrangements for the neck of that syringe barrel;

FIG. 2A shows a diagram of a syringe apparatus, comprising a syringe barrel such as that described with reference to FIG. 1;

FIG. 2B shows a three zoomed in views of a stopper of a syringe apparatus such as that illustrated in FIG. 2A;

FIG. 3 and FIG. 4 show a diagram of a dual-vial cartridge for delivery of a liquid drug product; and

FIG. 5 shows a diagram of a further dual-vial having a shoulder and indicating the use of a laser to provide weakening of the shoulder.

In the drawings like reference numerals are used to indicate like elements.

### SPECIFIC DESCRIPTION

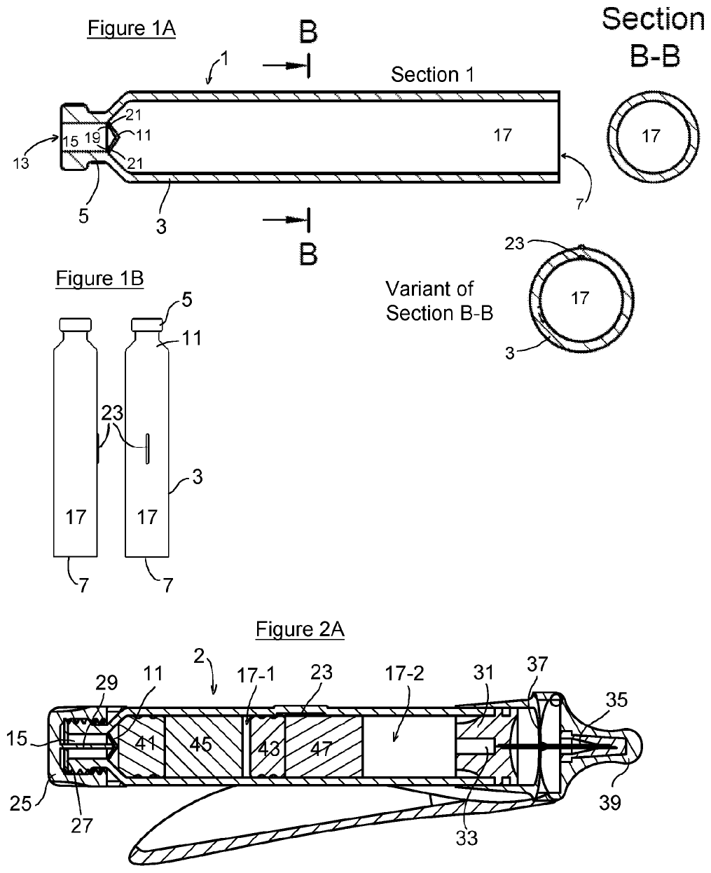

FIG. 1 shows a syringe barrel 1 for an auto-injector apparatus. The drawing comprises two section views of this syringe barrel. The first, labelled Section 1 in the drawing, comprises a longitudinal cross section of the syringe barrel, in other words a section through the plane comprising the long centre axis of the barrel. The second, labelled Section B-B in the drawing, comprises a view looking down the long centre axis of the barrel from the position marked B-B on Section 1. One possible variant of the Section B-B is also shown to illustrate an optional arrangement of a bypass channel.

The syringe barrel 1 is formed from a single integrated body 3 of glass. The body 3 is tube shaped, and has a neck 5 at one end, and a plain open end 7 at the other. The neck 5 may be narrower than the rest of the body 3, and is joined to the rest of the body by an external shoulder 9, which slopes from the neck 5 to the body 3.

Both ends of the tube shaped body 3 are open. Inside the body 3, a glass wall 11, formed integrally with the body 3 and the neck 5, blocks the base of the neck 5. The open end 13 of the neck 5 thus provides a propellant chamber 15 for holding a propellant, and which can be sealed by closing the open end 13 of the neck 5. The open end 7 of the body provides access to a medicament chamber 17 within the body 3. The medicament chamber 17 can be used for holding a piston and a substance to be delivered from the syringe barrel 1 by movement of the piston. The internal surface of the medicament chamber may comprise a groove, which may extend along the side wall of the medicament chamber 17 for providing a bypass around a syringe piston disposed in the medicament chamber. This can enable the barrel to be used in a dual syringe as described below.

The glass wall 11 separates this medicament chamber from the propellant chamber. As illustrated in FIG. 1, the glass wall 11 may be cone shaped, and tapering inward from the base of the neck 5 to an apex disposed further inside the tube shaped body 3. The surface of this cone shaped wall 11 may thus be disposed at an oblique angle to the opening at the end 13 of the neck 5, and thus provides an internal shoulder 19. This cone need not be complete, for example a shoulder 19 may be provided by an annular surface, for example at the outer boundary of the wall 11, where the wall meets the internal surface of the neck 5. This annular surface may be flat, and may be at right angles to the neck bore, or at an oblique angle to it, e.g. 45° as described elsewhere herein.

This internal shoulder 19 is weakened by a cleave line 21 which circumscribes a region of the glass wall 11, for example it may be disposed at the edge of the glass wall 11 adjacent the inside surface of the neck 5. Typically the cleave line 21 is provided by a series of regions of laser filamentation in the glass wall. This provides a ‘break zone’ in the form of a discontinuous line of weakened glass. However the weakening may also be provided scribing the surface such as with a laser, e.g. by performing laser ablation on the surface of the glass. Weakening could also be provided by printing a ceramic ink onto the glass wall, for example around a perimeter of the glass wall. The glass can then be annealed, e.g. in a Lehr process. Such ceramic ink may have a different coefficient of expansion from the glass, thereby to cause surface micro-cracks in the glass during the annealing process. The weakening may be inherent, for example it may arise from the shape of the glass wall and/or the thickness distribution of the glass.

Manufacture of the syringe barrel 3 described above will now be described. To begin, a length may be cut from a glass cylinder, e.g. using a heat cutter (such as a gas torch or laser) to provide a glass blank. Typically the cut end of this glass blank may be closed by the cutting process—e.g. one end of the cylinder may be closed by a surface of hot, soft, glass. Whilst still hot and soft, this flat closed end may then be deformed inwardly, into the interior of the glass blank, by a moulding tool. This may provide a glass cylinder with a concave glass bowl inside one end—this glass bowl may provide the glass wall 11 described above. Whilst this end of the cylinder and the glass are still hot and soft (or after additional heating) the neck 5 and external shoulder 9 of the syringe barrel 3 can be formed by pushing (e.g. by rolling) this hot end of the glass cylinder against an appropriate moulding tool. The neck may also be formed with either a thread or crimp fit (e.g. on its outer surface) at this stage. The steps of forming the inner wall, and the neck and flange may be performed in a single process, e.g. they may be done concurrently at the same station of a production line. However it is performed, the resultant structure is a syringe barrel which comprises a propellant chamber 15 in its neck and a glass wall 11 closing off the interior end of the neck 5 from the rest of the inside of the cylinder. This wall may have an upper surface, at the internal shoulder 19 where the wall meets the inside wall of the neck, which may be at an oblique angle to the opening of the neck, for example it may be cone shaped.

The weakening of the glass wall 11 may be created at this stage. This may be done by providing laser energy (e.g. from an ultra-short pulsed laser such as a picosecond or femtosecond laser) onto the internal shoulder 19 on the wall 11. The laser may be directed through the open end 13 of the neck 5, at an angle to the central axis of the tube that is chosen so that the laser can be normally incident, within ±10°, onto the upper surface of this shoulder 19 when directed through the open end 13 of the neck 5. This angle may be 45°, or another angle selected based on the neck bore diameter and the depth from the opening of the neck to the shoulder 19 to enable a laser to be normally incident (±10°) on the shoulder 19. The laser may be sufficiently energetic to cause weakening of the glass. The laser beam may be scanned across the surface of the wall (e.g. in a circular or closed path) to provide a break zone that, when broken, will cause part of the glass wall to detach from the neck. For example, the laser may be scanned around the circumference of the glass wall to create a circumferential break zone that is weaker than the rest of the wall. This may be achieved by holding the laser beam stationary and moving, for example rotating, the workpiece to scan the laser beam across the surface of the workpiece.

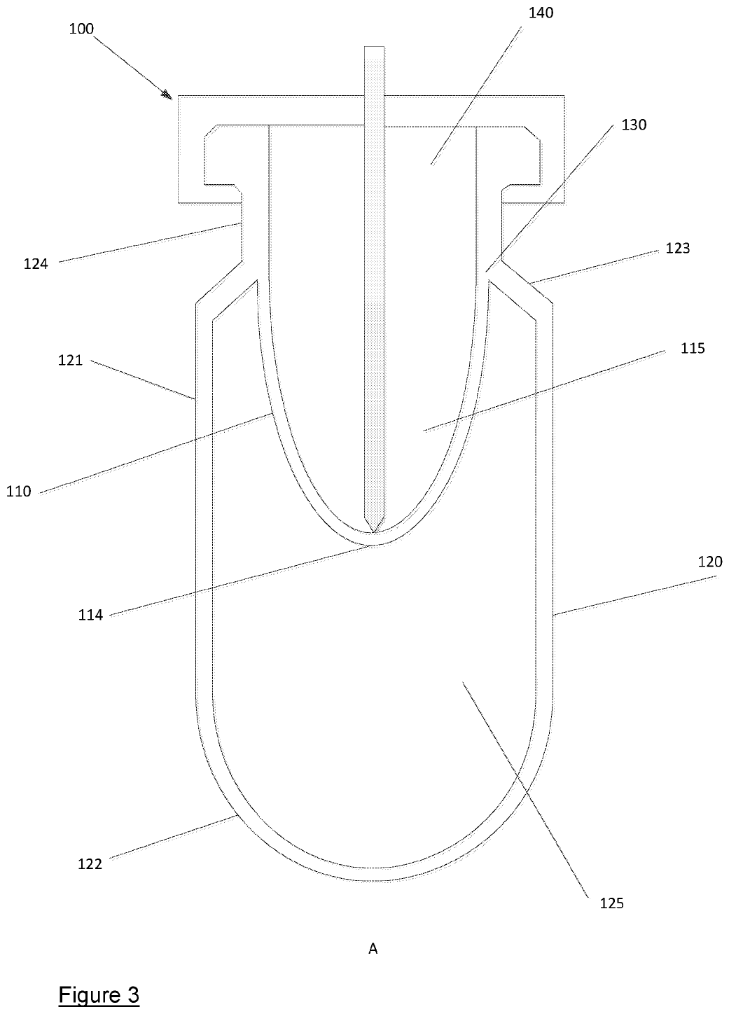

It will be appreciated in the context of the present disclosure that although the surface of the internal shoulder may be at an oblique angle to the opening of the neck, this is optional. For example the internal shoulder may be disposed at right angles to the neck bore. This may enable laser energy to be applied to the wall by directing a laser straight down the bore. In addition, the wall need not be cone shaped, for example the shoulder could be provided by a shelf or ridge around the edge of the wall, and the wall could be bowl shaped or vial shaped, for example as illustrated in FIG. 3 and described below.

FIG. 1B shows a side view of a syringe barrel such as that described with reference to FIG. 1A, and comprising a bypass channel such as that described with reference to the variant of Section B-B in FIG. 1. As illustrated in FIG. 1B, the bypass may comprise a groove which is aligned with the long axis of the medicament chamber 17. The bypass channel may be disposed at an intermediate position, between the wall 11 and the open end 7 of the medicament chamber 17. The barrel may comprise more than one such bypass channel, for example two or more such channels may be provided. These may be straight, as illustrated in FIG. 1B, or they may follow an arcuate path along the wall, for example they may be helical.

As noted above, the neck of the barrel may be narrower than the rest of the body, but as illustrated in the thumbnail sketches in the corner of FIG. 1B, the neck may have a variety of forms. For example it may comprise a flange, or threading for securing a cap to the neck. It may also be wider than the body.

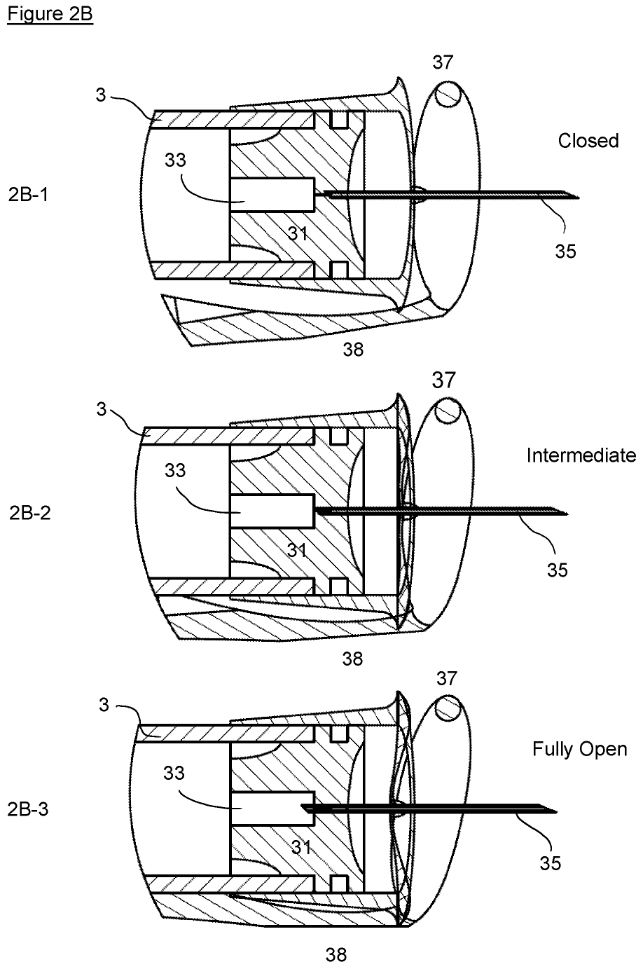

FIG. 2A shows a section through a syringe apparatus 2 comprising a syringe barrel 3′ such as that described above with reference to FIG. 1.

The syringe barrel of the apparatus illustrated in FIG. 2A is a barrel for a dual-chamber syringe which incorporates a bypass channel on an inside wall of the barrel. This can allow fluid to transfer from a rear chamber of the syringe past a piston to the front chamber of the syringe.

In addition to the syringe barrel 3′, the syringe apparatus 2 also comprises an actuator cap 25 covering the neck 5 of the syringe barrel 3′. The actuator cap is coupled to an actuator rod 29 which bears against the glass wall 11, so that by urging the actuator cap down onto the neck, the rod 29 can be used to break the glass wall 11. Inside the actuator cap 25, a propellant chamber cap 27 encloses the propellant chamber 15 to seal a propellant therein. An actuator 29 is disposed in the propellant chamber 15 between the propellant chamber cap 27 and the glass wall 11 of the propellant chamber 15.

The open end 7 of the medicament chamber 17 may be closed by a stopper 31 comprising a needle channel 33 for delivery of a substance from the medicament chamber 17 to a hypodermic needle 35. Also seated in the stopper 31 is a hypodermic needle 35, the internal lumen of which is connected to the needle channel 33. A needle hub 37 may also be provided to hold the hypodermic needle 35 in place in the stopper 31, and a needle shield 39 may be coupled to the needle hub 37 to cover the sharp end of the hypodermic needle.

A first piston 41 is disposed in the medicament chamber 17 of the syringe barrel 3′, adjacent the glass wall 11. The first piston 41 is cylindrical and surrounded about its circumference by at least one gasket which provides a seal between the piston 41 and the internal surface of the medicament chamber 17. A second piston 43, comprising a similar gasket arrangement, is also disposed in the medicament chamber 17 between the first piston 41 and the stopper 31.

A space 17-1 between the first piston and the second piston holds a first component 45 of a medicament. A space 17-2 between the second piston 43 and the stopper 31 holds a second component 47 of the medicament.

The syringe barrel 3′ also comprises a bypass channel 23, such as a longitudinal groove in the internal surface of the wall of the medicament chamber between the second piston and the stopper. The bypass channel may be longer than the second piston 43, but shorter than the first piston 41.

A safety cap may be provided, such as a rigid removable shield to prevent inadvertent operation of the actuator 29.

To use the apparatus, an operator would first remove such a cap from the neck to allow access to the actuator 29.

The actuator 29 may comprise a cap 25 and a rod which passes from the cap 25 through a sealed opening in the propellant chamber cap 27 for bearing upon the glass wall 11 between the propellant chamber 15 and the medicament chamber 17. The actuator 29 is operable, for example by pressing the cap 29 to break the glass wall 11 thereby to release the propellant to act upon the first piston 41. Upon actuation of the actuator 29, the propellant chamber cap 27 remains sealed to hold the propellant inside the barrel 3′. The propellant chamber cap 27 may be threaded or crimped onto the neck 5 of the barrel 3′. The actuator cap 25 may also be threaded on to the neck, so that screwing the actuator cap 25 down forces the actuator rod 29 onto the glass wall 11.

The first piston 41, and the second piston 43 are both moveable axially along the medicament chamber 17. The two pistons 41, 43 are arranged so that a proximal-distal movement of the first piston 41, axially along the medicament chamber 17, causes axial movement of the second piston 43 in the same direction. The second piston 43 is arranged so that, as it advances distally along the medicament chamber, it reaches a location at which the proximal end of the bypass channel 23 is proximal of the proximal end of the second piston 43 whilst the distal end of the bypass channel 23 is distal of the distal end of the second piston 43. When it is thus positioned, continued axial movement of the first piston 41 causes the first component 45 of the medicament to be expelled from the space 17-1 between the first piston 41 and the second piston 43 and into the space 17-2 between the second piston 43 and the stopper 31. This allows mixing of the two components 45, 47 of the medicament prior to their delivery from the syringe. The pistons 41, 43 are arranged so that, continued axial movement of the first piston from this position causes it to abut the proximal end of the second piston and to close the proximal end of the bypass channel, thereby to prevent escape of the propellant. The propellant can thus continue to push the two pistons distally along the medicament chamber for expelling the medicament through the needle channel into a lumen of the hypodermic needle.

To package a medicament in such an apparatus 2, a syringe barrel 3′ such as that illustrated in FIG. 1, but having a bypass channel in a wall of the medicament chamber 17, is provided.

The propellant chamber 15 is sealed with the propellant chamber cap 27 and actuator 29, and a propellant such as a hydrofluoroalkane (HFA) is provided into the propellant chamber 15, this may be done by a ‘cold-filling’ process wherein the propellant, packaging and filling environment temperature is reduced to below the propellant's boiling point (−16 to −26° C.) so the chamber 15 can be filled with the liquid and closed with 27. Alternatively it may be filled via a one-way valve or any appropriate means. Examples of HFA propellant include hydrofluoroalkane 134a (HFA 134) and hydrofluoroalkane 227a (HFA 227). Other propellants such as pressurised gases, for example N₂, CO₂, O₂ and other gases may also be used. Typically the fluid pressure of the propellant is between 2 bar and 10 bar dependent upon temperature.

The first piston 41 is then provided into the medicament chamber 17 so that its proximal end is adjacent to (e.g. abuts) the glass wall 11 of the propellant chamber 15. This may be done by inserting a tubular sleeve within the barrel 3′, down which the piston is pushed until it exits the bottom of the sleeve and expands against the barrel 3′.

A first component 45 of a medicament composition is then provided into the medicament chamber of the syringe barrel via the open end of the barrel. The second piston 43 is then provided into the medicament chamber to enclose the first component between the two pistons. This may be done by inserting a tubular sleeve within the barrel, down which the piston is pushed until it exits the bottom of the sleeve and expands against the barrel.

The open end 7 of the medicament chamber is then closed by pushing the stopper 31 into it. The needle hub 37, hypodermic needle 35, and needle shield 39 can then be put in place.

In operation, the actuator cap 25 enables operation of the actuator 29. The actuator 29 is then caused to press against the glass wall 11 which breaks it along the cleave line 21 to release the propellant from the propellant chamber 15. This causes the first piston 41 to move distally along the medicament chamber 17.

This in turn causes the second piston 43 to advance distally along the medicament chamber 17 to a position in which the bypass channel 23 allows fluid to flow around the second piston 43 into the space 17-2 between the second piston 43 and the stopper 31. The first piston 41 continues to advance, and to expel fluid through the bypass channel 23, until the first piston 41 abuts the proximal end of the second piston 43. Pressure from the propellant then causes the two pistons 41, 43, to continue to advance together for expelling fluid in the medicament chamber 17-2 (e.g. the mixed medicament) via the needle channel.

FIG. 2B shows a series of views of the end 7 of the barrel 3 and the stopper 31. The first of these views 2B-1 shows the apparatus in a closed configuration. The second view 2b-2 shows the apparatus in an intermediate, flexi-open, configuration. The third view 2b-3 shows the apparatus in an open configuration.

As illustrated in FIG. 2B, the end of the syringe apparatus 2 is closed by a stopper. A needle channel 33 is provided on the proximal side of the stopper (its inside surface) opposite to the needle 35 and the needle hub 37. The needle hub 37 may comprise a deformable portion 38 which can be deformed from a convex state, via an intermediate state, into a concave state. The deformable portion may be resilient, and may be arranged so that when in its intermediate state it will tend, resiliently, to return to its convex state if released. The deformable portion may be provided by a Belleville washer or any other appropriate structure capable of providing equivalent function.

The hypodermic needle 35 is affixed to the deformable portion 38 of the needle hub 37 to enable it to be moved axially into the stopper 31 toward the needle channel 33.

As illustrated in the closed configuration 2B-1 the deformable portion of the needle hub 37 is in its convex state, and the proximal end of the needle 35 terminates in the material of the stopper 31. It is thus separated from the needle channel, so the space 17-1 in the medicament chamber 17 remains closed.

In the intermediate configuration, the deformable portion is in its intermediate state, and the proximal end of the needle 35 has pierced the material of the stopper to enter the needle channel 33. If the deformable portion 38 is released, it will return to its closed configuration as illustrated in 2B-1. The material of the stopper may be resilient, so that if the proximal end of the needle is advanced distally, out of the channel 33, the material of the stopper 31 may at least partially reseal the hole pierced by the needle 35.

In the open configuration 2B-3, the deformable portion is in its concave configuration and so the needle is fixedly held with its proximal end in the needle channel 33.

This can enable the needle channel to be temporarily connected to the lumen of the hypodermic, e.g. to vent air from the space 17-1 in the barrel. The needle channel 33 can then be reclosed by allowing the deformable portion to return to its convex state. Then, when there is a need to deliver the content of the syringe, the deformable portion can be forced into its concave state so that the lumen of the needle 35 is held in the needle channel.

It will be appreciated in the context of the present disclosure that although this has been described with reference to a hypodermic needle, the same arrangement may also be used with any other dispensing lumen, such as a nozzle, provided that it is able to pierce the stopper upon movement of the deformable portion 38 of a hub 37.

FIG. 3 shows a diagram of a dual-vial cartridge for delivery of a liquid medicament.

FIG. 3 shows a dual vial 100 comprising a first vial 110 and a second vial 120 both of which may be integrally formed of a single continuous piece of material. This single continuous piece of material comprises glass which provides the walls and base of the first vial 110 and the walls and base of the second vial 120.

The first vial 110 is nested inside the second vial 120. This dual vial may comprise a medicament in one compartment (such as the first vial 110), and a propellant in the other. An actuator comprising an outlet channel may be operable to break a glass wall between the two, thereby to expel the medicament through an outlet channel in the actuator. Of course, the actuator and the outlet channel may be provided separately.

A transition zone 130 is illustrated in FIG. 1 as the region of the single piece of material which joins the first vial 110 to the second vial 120. The transition zone 130 forms a coupling point or join between the first vial 110 and the second vial 120.

The first vial 110 has a closed end 114 and an open end. The open end may provide a mouth 140 for the first vial through which a substance can be provided into the internal volume 115 of the first vial 110. The first vial 110 may be closed (as illustrated in FIG. 2) by covering or otherwise occluding the mouth 140 to completely enclose its internal volume, for example it may be sealed. The first vial may be used to hold a propellant such as any of those described herein.

The first vial 110 is substantially cylindrical in that it is circular in cross-section, although it may have a rounded taper (e.g. be a test-tube bottom shape) as illustrated in FIG. 1.

For example, from the closed end 114 of the first vial, the diameter of the first vial 110 may increase. The first vial may be concentric (e.g. co-axial) with the second vial 120. The part of the single piece of material which provides the first vial 110 may include the closed end 114 and the mouth 140, at which point it meets the transition zone 130. On the other side of the transition zone 130, the single piece of material may continue and provide the second vial 120.

The diameter of the first vial 110 is less than that of the second vial 120 until, at the transition zone 130, the two vials 110, 120 meet.

The second vial 120 may have the same shape as the first vial 110. The second vial 120 encloses a second volume 125 which surrounds the first vial 110. As illustrated in FIG. 1 the second vial 120 has a lower portion 122, which is further away from the mouth 140 than the closed end 114 of the first vial 110. A body 121 of the second vial is connected by a shoulder 123 to the transition zone 130.

The second vial may also include a neck 124 connected to the body 121 by the shoulder 123. The neck 124 and/or the shoulder 123 of the second vial 120 may surround the mouth 140 of the first vial 110. In the example illustrated in FIG. 1, the second vial 120 is substantially cylindrical in that it is circular in cross section (in the plane perpendicular to the longitudinal axis). The mouth 140 and the body 121 of the second vial 120 are largely cylindrical in shape. The shoulder 123 tapers in from the body 121 to the neck 124, and the lower portion 122 may also taper in to a closed end of the second vial 120. For example, the shoulder 123 may taper outward (increase in diameter) from the region of the neck 124 and transition zone 130 to the body 121. The lower portion 122 may taper inward (decrease in diameter) from the body 121 to a heat seal or closure of the second vial 120. As mentioned above, the taper of the lower portion may be rounded or test-tube bottom shaped.

At the transition zone 130, the portion of the single piece of material providing the first vial 110 is connected to the portion providing the second vial 120 around the entire circumference of the join between the mouth 140 of the first vial 110 and the shoulder 123 and/or neck 124 of the second vial. The wall of the first vial 110 is continuous (has no holes, breaks or cracks in) for the entirety of the first vial 110. In this context, the first vial 110 seals the second vial 120 as the piece of material providing the first vial 110 completely obstructs a path from the mouth 140 to the second volume 125. The first vial 110 may provide a seal for the second vial 120. The second vial 120 may provide an ampoule sealed by the first vial 110 and the lower portion 122 of the second vial 120. As the first vial 110 and the second vial 120 are connected at the transition zone 130, which encloses the mouth 140, the two vials may be thought of as sharing the mouth 140, i.e. they have a common mouth.

The first vial 110 may be tapered so that it is generally test-tube bottom-shaped, i.e. it has a rounded end. The second vial 120 may also be tapered so that it is generally test-tube bottom-shaped. The single continuous piece of material providing the first vial 110 and the second vial 120 is glass.

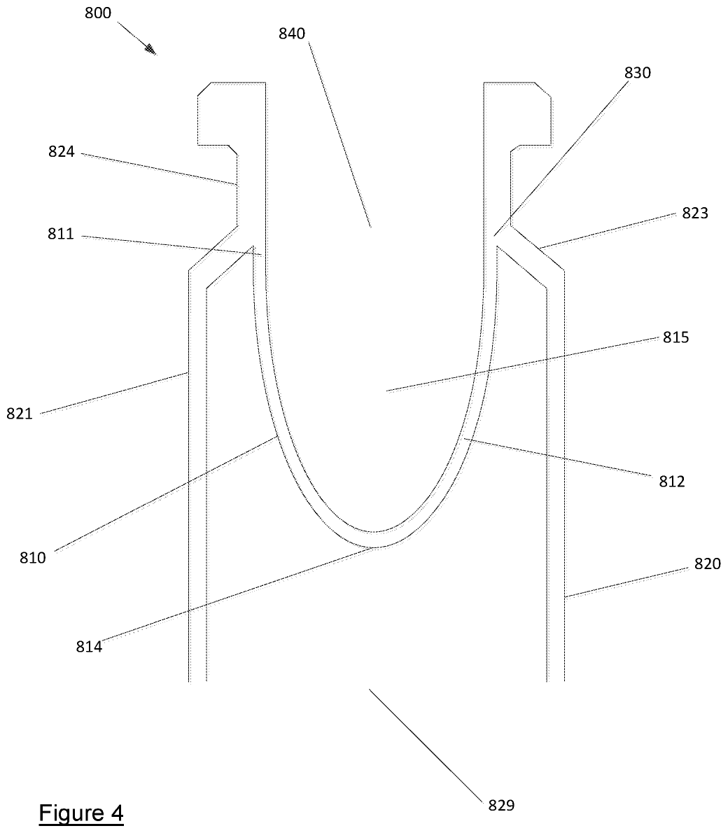

Manufacture of the dual vial described above will now be described with reference to FIG. 4. To manufacture a dual vial as described above, a length may be cut from a glass cylinder, e.g. using a heat cutter (such as a gas torch or laser) to provide a glass blank. Typically the cut end of this glass blank may be closed by the cutting process—e.g. one end of the cylinder may be closed by a flat surface of hot, soft, glass. Whilst still hot and soft, this flat closed end may then be deformed inwardly, into the interior of the glass blank, by a moulding tool. This may provide a glass cylinder with a concave glass bowl inside one end—this glass bowl may provide a first vial 810 of a dual vial (e.g. as described above). Whilst this end, and the first vial 810, are still hot and soft (or after additional heating) the neck 824 and shoulders 823 of the dual vial described above can be formed by pushing (e.g. by rolling) this hot end of the glass cylinder against an appropriate moulding tool. The neck may also be formed with either a thread or crimp fit (e.g. on its outer surface) at this stage. The steps of forming the inner wall, and the neck and flange may be performed in a single process, e.g. they may be done concurrently at the same station of a production line. The However it is performed, the he resultant structure is a vial blank which comprises the neck 824 and shoulders 823 of a dual vial (e.g. as described above with reference to FIG. 3), and the first vial 810. Joined to the neck 824 and shoulders 823, and surrounding the first vial 810, is the rest of the glass cylinder which will provide the body 820 and optionally a lower portion of the second vial 820. This cylinder may be open at the other end (to provide an open end 829) at this stage, as illustrated in FIG. 4.

The break zone may be created at this stage. This may be done by providing laser energy (e.g. from an ultra-short pulse laser such as a picosecond or femtosecond laser) onto a region of the wall of the first vial 810. The laser may be sufficiently energetic to cause weakening of the glass. The laser beam may be scanned across the surface of the wall (e.g. in a circular or closed path) to provide a break zone that, when broken, will cause all or part of the first vial 810 to detach from the neck 824 and shoulders 823 of the dual vial 800. For example, the laser may be scanned around the circumference of the first vial 810 to create a circumferential break zone that is weaker than the rest of the wall of the first vial.

To package a two component composition into this dual vial 800, it can be inverted so that the neck 824 and shoulders 823 point downward, and the open end 829 of the glass cylinder points upward. A component of this two component composition can then be introduced into the cylinder. The open end 829 of the glass cylinder can then be heat sealed, e.g. in the manner of an ampoule. For example a gas torch or laser may be used to heat seal the glass cylinder to provide the lower end of the second vial. The dual vial can then be inverted and the other component of the two component composition can be introduced into the first vial 810 (e.g. through the mouth of the neck). The first vial 810 can then be closed—e.g. by crimping or threading an appropriate cap onto the neck.

The cap may comprise an actuator for acting on the wall of the inner vial to break it off at the break zone and mix the two components. The cap may also comprise a selectively operable valve, actuable to allow pressurised fluid inside the vial to flow out of the vial. One of these two components may comprise a propellant, such as any of those described herein, in which case typically the propellant is introduced into the first vials described above.

Of course, the first vial 810 may be filled and closed first, before the second vial 820 is filled and heat sealed. The sequence of these two operations may be significant in some circumstances.

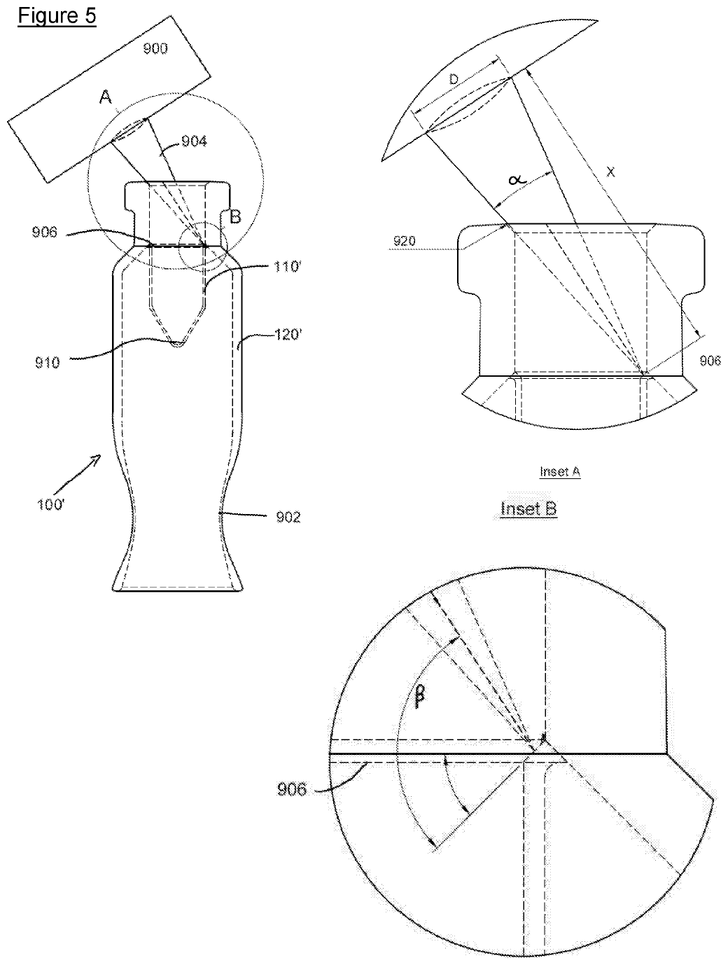

FIG. 5 shows a diagram of another example of a vial such as that described above with reference to FIG. 3.

FIG. 5 comprises a view of the entire vial, and two insets.

Inset A shows the neck of the dual vial, and the location of the shoulder which joins the inner (first) vial to the outer (second) vial. Inset B shows more detail of the shoulder. The view of the entire vial also illustrates the use of a laser to weaken the shoulder and provide a cleave line.

The dual vial 100′ illustrated in FIG. 4 comprises a first vial 110′ and a second vial 120′ both of which may be integrally formed of a single continuous piece of material, such as glass. This single continuous piece of material provides the walls and base of the first vial 110′ and the walls and base of the second vial 120′. As illustrated in FIG. 4, the bottom of the second vial 120′ may be open to enable it to be filled with a material to be packaged. The second vial 120′ may comprise a waist portion, in which the walls of the vial are narrowed and/or a flared end. This may provide an ampoule-like “drawn stem” to aid in a flame sealing process, which can be used to enclose a substance in the second vial, surrounding the first vial.

The first vial 110′ is nested inside the second vial 120′, and the first vial 110′ is closed at one end 910, inside the second vial 120′. The other end of the first vial 110′ is open. This open mouth of the first vial 110′ is joined by an annular shoulder 906 around its periphery to the internal surface of the second vial 120′. The inward facing surface of this annular shoulder may be sloping and may be at least partially flat. For example it may provide an open ended frusto-conical form, the wider end of which may be joined to the internal surface of the second vial (e.g. inside its neck), and the narrower end of which may provide the mouth of the first vial 110′.

As illustrated in Inset A, laser optics 900 may be arranged to direct laser energy 904 on to the shoulder 906. The optics 900 may be arranged to focus the laser on the shoulder 904 of the dual vial 100′. The beam 904 may have a width D as it leaves the optics, and a convergence angle α, which may be chosen based on the distance X between the optics and the shoulder—for example it may be chosen based on the length of the neck and the width of the open end of the neck. The angle between the axis of the beam and the surface of the shoulder 04 is denoted, β in Inset B.

As shown in Inset A, the inside edge 920 of the mouth of the second vial 120′ (e.g. the open end of the neck of the dual vial 100′) may be bevelled. For example it may have a sloping surface rather than a sharp corner. This may assist in the delivery of laser light to the shoulder by making the mouth wider than the rest of the neck. The tapering of the beam 904 by the laser optics 900 may also assist in this regard because it may reduce the tendency of the beam envelope to hit the wall of neck. This can help to enable the laser to be normally incident (e.g. at right angles to) a flat surface of the shoulder. This can aid the process of modifying the material of the wall with the laser light to create defects which weaken the wall (e.g. by laser filamentation). Counterintuitively, the tapering of the laser beam may also promote normal incidence of the laser on the shoulder because provided that the misalignment from normal incidence is less than the convergence angle of the beam, at least a portion of the laser will be normally incident. As an example, if the convergence angle α of the beam is 12°, then the angle β between the axis of the beam and the shoulder may be as large as 102° (or as small as 78°) whilst still providing at least some normally incident light.

The vial 100′ may comprise a single continuous piece of material, such as glass, arranged to provide both the first vial 110′ and the second vial 120′. For example the entire vial 110′ may be integrally formed from the same single piece of glass.

Further embodiments are envisaged. For example the bypass channel and second piston of the syringe barrels and syringe apparatus described above are optional—apparatus of the present disclosure may be used to provide auto-injectors for single component medicaments.

The systems and apparatus of the present disclosure need not be used with needles. For example, the outlet of the syringe barrel may comprise a nozzle, e.g. for oral or intra-nasal delivery of medicaments.

The syringe barrels described and claimed herein may comprise a single continuous piece of material arranged to provide both the propellant chamber and the medicament chamber, and the glass wall therebetween. For example the entire syringe barrel may be integrally formed from the same single piece of glass.

The weakening of the glass wall (such as that on the shoulder of the vial 110′) may be provided by a break zone which is weaker than the rest of the wall. The break zone may comprise a cleave line which circumscribes a region of the glass wall. For example, it may be arranged in a closed path, such as a band around the circumference of the glass wall. This closed path need not be continuous in the sense of being uninterrupted, for example it may be discontinuous in the manner of a line of perforations, such as those which surround postage stamps. It will be appreciated in the context of the present disclosure however that such weakening may be provided without puncturing the glass wall. It may however be arranged in a closed loop path.

The weakening may be done by chemical etching, or by applying laser energy to the break zone, or by any other method of weakening such as scoring the glass wall. The application of laser energy for this purpose may have some advantages. This may cause laser filamentation resulting in material modification of glass within the wall. Laser energy may also be applied to cause surface ablation to scribe or score the wall. The laser energy may be applied to cause heating and thermal expansion to create internal stress within the wall. Laser energy may be applied to the wall by a laser directed through an opening of the neck of the barrel.

Laser energy may be provided by an ultrashort pulse laser. It is to be appreciated in the context of this disclosure that an ultrashort pulse laser may include a femtosecond or picosecond laser.

To the extent that certain methods may be applied to the living human or animal body, it will be appreciated that such methods may not provide any surgical or therapeutic effect. In addition, it will be appreciated that such methods may be applied ex vivo, to tissue samples that are not part of the living human or animal body. For example, the methods described herein may be practiced on meat, tissue samples, cadavers, and other non-living objects.

With reference to the drawings in general, it will be appreciated that schematic functional block diagrams are used to indicate functionality of systems and apparatus described herein. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Any apparatus feature as described herein may also be provided as a method feature, and vice versa. Furthermore, equivalents and modifications not described above may also be employed without departing from the scope of the invention, which is defined in the accompanying claims.

1. A single use syringe barrel comprising:
a single integrated glass body forming a medicament chamber, a propellant chamber, and a glass wall separating the medicament chamber from the propellant chamber.

2. The single use syringe barrel of claim 1, wherein the glass wall is weakened.

3. The single use syringe barrel of claim 2, wherein the weakening comprises a cleave line which circumscribes a region of the glass wall.

4. The single use syringe barrel of claim 3 wherein the cleave line comprises laser filamentation of the glass wall.

5. The single use syringe barrel of claim 3 or 4 wherein the cleave line is discontinuous.

6. The single use syringe barrel of any preceding claim wherein the glass wall comprises a shoulder, disposed at an oblique angle to an opening of the glass body.

7. The single use syringe barrel of claim 6 wherein the weakening is disposed on the shoulder.

8. The single use syringe barrel of any preceding claim, wherein the glass body is a tube, and the propellant chamber is disposed at a first end of the tube.

9. A syringe comprising:
a medicament chamber having an outlet to enable a medicament to be expelled from the medicament chamber;
a first piston movable to expel medicament from the medicament chamber via the outlet;
a propellant chamber having a glass wall breakable to release propellant to move the first piston to expel said medicament.

10. The syringe of claim 9 wherein the medicament chamber, the glass wall, and the propellant chamber are integrally formed.

11. The syringe of claim 10 wherein the glass wall is weakened by a cleave line which circumscribes a region of the glass wall.

12. The syringe of claim 11 wherein the cleave line comprises laser filamentation of the glass wall.

13. The syringe of claim 11 or 12 wherein the cleave line is discontinuous.

14. The syringe of any of claims 9 to 13 wherein the syringe is a tube and the glass wall comprises a shoulder surface, disposed at an oblique angle to an end of the tube.

15. The syringe of claim 14 wherein the weakening is provided in the shoulder surface.

16. The syringe of any of claims 11 to 15 wherein the propellant chamber comprises a movable member, actuable to break the glass wall.

17. The syringe of any of claims 9 to 16 comprising a second piston disposed in the medicament chamber, and a bypass channel in a sidewall wall of the medicament chamber between the second piston and the outlet.

18. The syringe of claim 17 comprising a first component of a medicament composition disposed between the first piston and the second piston, and a second component of the medicament composition disposed between the second piston and the outlet.

19. The syringe of any of claims 9 to 18 comprising a propellant in the propellant chamber.

20. A method of packaging a medicament, the method comprising:
providing a syringe barrel having a propellant chamber having a glass wall, wherein a first piston is disposed in the syringe barrel;
and providing a first medicament into the syringe barrel via an open end of the barrel, between the first piston and the open end.

21. The method of claim 20, further comprising, after providing the medicament, closing the open end with a stopper having an outlet for delivery of the medicament from the syringe.

22. The method of claim 21 wherein the outlet comprises a connection for connecting the stopper to a hypodermic needle.

23. The method of any of claims 20 to 22 comprising providing a propellant into the propellant chamber.

24. The method of any of claims 20 to 23 wherein the syringe barrel comprises a bypass channel, the method further comprising providing a second piston into the barrel, past the bypass channel to enclose the first medicament, and providing a second medicament into the barrel after the second piston.

25. The method of any of claims 20 to 22 wherein the syringe barrel comprises the syringe barrel of any of claims 1 to 8.

26. A dual vial comprising a first vial and a second vial, wherein the first vial is nested inside the second vial;
the first vial comprising a closed end inside the second vial and an open end, wherein the open end of the first vial is joined to an internal surface of the second vial by a shoulder which surrounds the open end of the first vial;
wherein the shoulder is annular and comprises a flat sloping surface.

27. The dual vial of claim 26, wherein the shoulder comprises defects to provide a cleave line for separating the first vial from the second vial.

28. The dual vial of claim 26 or 27 wherein the flat sloping surface is positioned within a neck of the second vial and faces toward an opposite edge of an open end of the neck.

29. The dual vial of claim 28, wherein the neck is cylindrical and has a longitudinal axis, wherein the flat sloping surface is arranged at an angle of at least 10° to the longitudinal axis, for example at least 30°, for example less than 80°, for example less than 60°, for example 45°.

30. A method of creating a cleave line in a dual vial,
the dual vial comprising a first vial and a second vial, wherein the first vial is nested inside the second vial and an open end of the first vial is joined to an internal surface of the second vial by a shoulder which surrounds the open end of the first vial,
the method comprising directing laser light onto the shoulder through an open end of the second vial.

31. The method of claim 30 wherein the shoulder is annular and comprises a flat sloping surface.
